SEQUENCE LISTING (30 sequences)

SEQ ID NO: 1 - DNA, 19 bp, Artificial Sequence
  36b4 cDNA-189 forward primer
agatgcagcagatccgcat

SEQ ID NO: 2 - DNA, 19 bp, Artificial Sequence
  36b4 cDNA-247 reverse primer
gttcttgcccatcagcacc

SEQ ID NO: 3 - DNA, 18 bp, Artificial Sequence
  36b4 Taqman probe
cgctccgagggaaggccg

SEQ ID NO: 4 - DNA, 28 bp, Artificial Sequence
  mABCA1 cDNA-6484 forward primer
ggtttggagatggttatacaatagttgt

SEQ ID NO: 5 - DNA, 17 bp, Artificial Sequence
  mABCA1 cDNA-6577 reverse primer
cccggaaacgcaagtcc

SEQ ID NO: 6 - DNA, 25 bp, Artificial Sequence
  mABCA1 Taqman probe
cgaatagcaggctccaaccctgacc

SEQ ID NO: 7 - DNA, 24 bp, Artificial Sequence
  mTNF-alpha cDNA-243 forward primer
atcatcttctcaaaactcgagtga

SEQ ID NO: 8 - DNA, 19 bp, Artificial Sequence
  mTNF-alpha cDNA-368 reverse primer
ttgagatccatgccattgg

SEQ ID NO: 9 - DNA, 23 bp, Artificial Sequence
  mTNF-alpha Taqman probe
agcccacgtcgtagcaaaccacc

SEQ ID NO: 10 - DNA, 19 bp, Artificial Sequence
  MMP-9 forward primer
tcaccttcacccgcgtgta

SEQ ID NO: 11 - DNA, 17 bp, Artificial Sequence
  MMP-9 reverse primer
gtcctccgcgacaccaa

SEQ ID NO: 12 - DNA, 25 bp, Artificial Sequence
  MMP-9 probe
acccgaagcggacattgtcatccag

SEQ ID NO: 13 - DNA, 18 bp, Artificial Sequence
  iNOS forward primer
gcagctgggctgtacaaa

SEQ ID NO: 14 - DNA, 19 bp, Artificial Sequence
  iNOS reverse primer
agcgtttcgggatctgaat

SEQ ID NO: 15 - DNA, 18 bp, Artificial Sequence
  iNOS probe
tcaaaggtctcacaggct

SEQ ID NO: 16 - DNA, 23 bp, Artificial Sequence
  IL-6 forward primer
ctgcaagagacttccatccagtt

SEQ ID NO: 17 - DNA, 19 bp, Artificial Sequence
  IL-6 reverse primer
gaagtagggaaggccgtgg

SEQ ID NO: 18 - DNA, 26 bp, Artificial Sequence
  IL-6 probe
tgtcaccagcatcagtcccaagaagg

SEQ ID NO: 19 - DNA, 21 bp, Artificial Sequence
  IL-1beta forward primer
agaagctgtggcagctacctg

SEQ ID NO: 20 - DNA, 22 bp, Artificial Sequence
  IL-1beta reverse primer
ggaaaagaaggtgctcatgtcc

SEQ ID NO: 21 - DNA, 25 bp, Artificial Sequence
  IL-1beta probe
tctttcccgtggaccttccaggatg

SEQ ID NO: 22 - DNA, 21 bp, Artificial Sequence
  IL-1-RN forward primer
tgtttagctcacccatggctt

SEQ ID NO: 23 - DNA, 20 bp, Artificial Sequence
  IL-1-RN reverse primer
tcttgcagggtcttttccca

SEQ ID NO: 24 - DNA, 20 bp, Artificial Sequence
  IL-1-RN probe
agaggcagcctgccgccctt

SEQ ID NO: 25 - DNA, 20 bp, Artificial Sequence
  CD-68 forward primer
caaggtccagggaggttgtg

SEQ ID NO: 26 - DNA, 27 bp, Artificial Sequence
  CD-68 reverse primer
cggtacccatccccacctgtctctctc

SEQ ID NO: 27 - DNA, 24 bp, Artificial Sequence
  CD-68 probe
ccaaaggtaagctgtccataagga

SEQ ID NO: 28 - DNA, 25 bp, Artificial Sequence
  MCP-1 forward primer
agaagctgtgatcttcaagaccatt

SEQ ID NO: 29 - DNA, 20 bp, Artificial Sequence
  MCP-1 reverse primer
tgcttgtccaggtggtccat

SEQ ID NO: 30 - DNA, 25 bp, Artificial Sequence
  MCP-1 probe
ccaaggagatctgtgctgaccccaa

(19) United States
(12) Patent Application Publication
(10) Pub. No.: US 2004/0259948 A1
(43) Pub. Date: Dec. 23, 2004

(54) Reciprocal regulation of inflammation and lipid metabolism by liver X receptors

(76) Inventors: Peter Tontonoz, Sherman Oaks, CA (US); Sean B. Joseph, San Diego, CA (US); Antonio Castrillo, Los Angeles, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/755,720

(22) Filed: Jan. 12, 2004

Related U.S. Application Data
(60) Provisional application No. 60/439,570, filed on Jan. 10, 2003.

(51) Int. Cl. A61K 31/20

(52) U.S. Cl. 514/560

(57) ABSTRACT

The present invention is related to the role of liver X receptors (LXRs) in inflammation and immunity. More particularly, methods are disclosed for identifying and using LXR agonists for the treatment of inflammatory processes.

### RELATED APPLICATIONS

This application claims the benefit of U.S. Provisional Application No. 60/439,570, filed Jan. 10, 2003.

### FIELD OF THE INVENTION

The present invention is related to the role of liver X receptors (LXRs) in inflammation and immunity.

### BACKGROUND OF THE INVENTION

Inflammation is a complex stereotypical reaction of the body expressing the response to damage of its cells and vascularized tissues. Inflammatory responses can be divided into the following categories: allergic (reaginic) inflammation, inflammation mediated by cytotoxic antibodies, inflammation mediated by immune complexes, delayed-type hypersensitivity reactions. The following cells participate in inflammation: mast cells and basophils; eosinophils; neutrophils, and macrophages and monocytes.

The list of inflammatory diseases includes but not limited to rheumatoid arthritis, juvenile rheumatoid arthritis, systemic lupus erythematosus, osteoarthritis, degenerative joint disease, one or more connective tissue diseases, ankylosing spondylitis, bursitis, Sjogren's syndrome, psoriasis, psoriatic arthritis, neuralgia, synoviitis, glomerulonephritis, vasculitis, sarcoidosis, atherosclerosis, asthma, inflammatory bowel disease, inflammations that occur as sequellae to influenza, the common cold and other viral infections, such as, for example, hepatitis, gout, contact dermatitis, low back and neck pain, dysmenorrhea, headache, toothache, sprains, strains, myositis, burns, injuries, and pain and inflammation that follow surgical and dental procedures in a patient.

Non-steroidal anti-inflammatory drugs (NSAIDs) are widely used to treat inflammation. These exert anti-inflammatory, analgesic and antipyretic activity.

NSAID's include salicylates such as aspirin, sodium salicylate, choline salicylate, salicylsalicylic acid, diflunisal, and salsalate; indoleacetic acids such as indomethacin and sulindac; pyrazoles such as phenylbutazone, oxyphenbutazone; pyrrolealkanoic acids such as tolmetin; phenylacetic acids such as ibuprofen, feroprofen, flurbiprofen, and ketoprofen; fenamates such as mefanamic acid, and meclofenamate; oxicams such as piroxicam; and naphthaleneacetic acids such as naproxen. Nearly all NSAIDs act by inhibiting the activity of the cyclooxygenase-2 enzyme (COX-2). Aspirin, for example, acetylates and irreversibly inactivates cyclooxygenase. Others, such as indomethacin, inhibit cyclooxygenase activity reversibly by binding in a stereospecific manner to one or another of the subunits of the enzyme. NSAIDs are active in reducing the prostaglandin-induced pain and swelling associated with the inflammation process because they inhibit the cyclooxygenase-2 (COX-2) enzyme.

Most NSAIDs do not selectively inhibit only the COX-2 enzyme, but also inhibit the cyclooxygenase-1 (COX-1) enzyme, which is important in other biologically beneficial prostaglandin-regulated processes that are not associated with the inflammation process. The use of high doses of non-selective NSAIDs can produce severe side effects, including life-threatening ulcers, that limit their therapeutic potential. The new class of selective COX-2 inhibitors, such as rofecoxib, etoricoxib, celecoxib, parecoxib and valdecoxib, generally do not have the same side effects that are exhibited by the earlier NSAIDs.

Adrenal corticosteroids, which are alternatives to NSAIDs for treating inflammatory diseases, also have potentially severe side effects, especially when long-term therapy is involved. These steroids, including hydrocortisone, prednisolone, 6-alpha-methylprednisolone, triamcinolone, dexamethasone and betaroethasone, affect inflammation by a possible mechanism whereby they bind to intracellular glucocorticoid receptors to subsequently initiate a series of cellular events involving synthesis of new phospholipid inhibitory proteins, or lipocortins, that can affect the inflammatory and the teratogenic responses of certain cells exposed to glucocorticoids. The anti-inflammatory effect of glucocorticoids has been well documented.

Because existing anti-inflammatory agents and treatment regimens, such as NSAIDs, selective COX-2 inhibitors and glucocorticoids, may exhibit non-specific and potentially serious side effects, there remains a need for new compounds and therapeutic methods that reduce and/or prevent inflammation via alternate mechanisms of action, as well as screening assays for the discovery of such anti-inflammatory compounds. Accordingly, the present inventors have developed such screening assays and elucidated a new role for LXR receptors in the treatment and prevention of inflammatory conditions.

### SUMMARY OF THE INVENTION

The present invention is a method for treating an inflammatory disease in a mammalian patient, where the method comprises the step of treating the patient with an LXR agonist. Such diseases include, but are not limited to, rheumatoid arthritis, juvenile rheumatoid arthritis, systemic lupus erythematosus, osteoarthritis, degenerative joint disease, one or more connective tissue diseases, ankylosing spondylitis, bursitis, Sjogren's syndrome, psoriasis, psoriatic arthritis, neuralgia, synoviitis, glomerulonephritis, vasculitis, sarcoidosis, atherosclerosis, asthma, inflammatory bowel disease, inflammations that occur as sequellae to influenza, the common cold and other viral infections, such as, for example, hepatitis, gout, contact dermatitis, low back and neck pain, dysmenorrhea, headache, toothache, sprains, strains, myositis, burns, injuries, and pain and inflammation that follow surgical and dental procedures in a patient. Diseases that are likely to be responsive to this treatment include rheumatoid arthritis, juvenile rheumatoid arthritis, systemic lupus erythematosus, osteoarthritis, degenerative joint disease, one or more connective tissue diseases, ankylosing spondylitis, bursitis, Sjogren's syndrome, psoriasis, psoriatic arthritis, neuralgia, synoviitis, glomerulonephritis, vasculitis, sarcoidosis, atherosclerosis, asthma, inflammatory bowel disease, inflammations that occur as sequellae to influenza, the common cold and other viral infections, such as, for example, hepatitis, gout, contact dermatitis, low back and neck pain, dysmenorrhea, headache, toothache, sprains, strains, myositis, burns, injuries, and pain and inflammation that follow surgical and dental procedures in a patient.

The invention also is a method for treating an inflammatory disease in a patient which comprises treating the patient with a combination comprising an LXR agonist and an NSAID, which may be non-selective or may be a selective cyclooxygenase-2 (COX-2) inhibitor. Such diseases include, but are not limited to, the diseases listed above.

The invention also is a method for treating an inflammatory disease in a patient which comprises treating the patient with a combination comprising an LXR agonist and a corticosteroid. Such diseases include, but are not limited to, the diseases listed above.

The invention is also a method for inhibiting or preventing secondary inflammation in a patient developing inflammation at a primary site which comprises treating the patient with a composition which comprises an LXR agonist.

We demonstrate here that liver X receptors (LXRs) and their ligands are negative regulators of macrophage inflammatory gene expression. Transcriptional profiling of lipopolysaccharide (LPS)-induced macrophages reveals reciprocal LXR-dependent regulation of genes involved in lipid metabolism and the innate immune response. In vitro, LXR ligands inhibit the expression of inflammatory mediators such as inducible nitric oxide synthase (iNOS), cyclooxygenase 2 (COX-2) and interleukin-6 in response to bacterial infection or LPS stimulation. In vivo, LXR agonists reduce inflammation in a model of contact dermatitis and inhibit inflammatory gene expression in the aortas of atherosclerotic mice. These findings identify LXRs as lipid-dependent regulators of inflammatory gene expression that are envisioned to serve to link lipid metabolism and immune functions in macrophages, and provide a target for regulating inflammation.

More particularly, in one embodiment of the present invention, a method is disclosed for treating an inflammatory condition in a subject comprising administering to the subject an amount of an agent sufficient to inhibit expression of a gene encoding an inflammatory mediator. In preferred embodiments, the gene encoding the inflammatory mediator is selected from the group consisting of NOSi, COX-2 and IL-6, IL-1β granulocyte colony-stimulating factor (G-CSF), chemokines (such as monocyte chemoattractant protein-i (MCP-1), MCP-3, and macrophage inflammatory protein-1β (MIP-1β) interferon-inducible protein-10 (IP-10), and the metalloproteinase MMP-9.

In another preferred embodiment, a method is disclosed for treating or preventing a macrophage-dependent inflammatory disease in a subject, comprising administering to the subject a therapeutically effective amount of an agonist of a liver X receptor (LXR). The macrophage-dependent inflammatory disease may be atherosclerosis, rheumatoid arthritis, or glomerulonephritis. The agonist is preferably selected from the group consisting of GW3965 and T1317.

In a variation, a method is disclosed for inhibiting macrophage-dependent inflammatory gene expression in a mammal. The method comprises administering to the mammal an amount of an agonist of a liver X receptor (LXR) sufficient to antagonize NF-κB signaling such that the macrophage-dependent inflammatory gene expression is reduced.

In another variation, a method is disclosed for reducing an inflammation in a mammal, comprising administering to the mammal a therapeutically effective amount of an agonist of a liver X receptor (LXR).

In another aspect, the present invention relates to a method of enhancing lipid metabolism and cholesterol clearance, while reducing inflammation in a mammal. The method comprises administering to the mammal a therapeutically effective amount of an agonist of a liver X receptor (LXR).

### BRIEF DESCRIPTION OF THE DRAWINGS

FIG. 1 (A-E) shows that synthetic liver X receptor (LXR) ligand (GW3965) inhibits the macrophage response to bacterial pathogens. a, LXR agonist inhibits nitrite production in response to bacterial infection. b, LXR agonist inhibits induction of nitric oxide synthase (iNOS) mRNA in response to bacterial infection. c, LXR agonist inhibits induction of nitric oxide synthase (iNOS) mRNA in response to lipopolysaccharide (LPS) stimulation. d, Dose-dependent inhibition of nitrite production by GW3965. e, Reciprocal dose-dependent regulation of ABCA1 and iNOS mRNA expression by GW3965. White bars—ABCA1 mRNA; Black bars—iNOS mRNA.

FIG. 2 shows that synthetic LXR ligand inhibits the expression of many genes involved in inflammation in macrophages. In contrast, genes involved in lipid metabolism are induced by LXR ligand. Transcriptional profiling of LXR agonist effects in LPS-activated macrophages.

FIG. 3 shows that synthetic LXR ligand inhibits the expression of iNOS, COX-2, IκBα, IL-1β, MCP-1 and IL-6 in macrophages. a, Receptor-dependent inhibition of iNOS, COX-2 and IκBα mRNA expression by LXR ligand (GW3965). b, Receptor-dependent inhibition of IL-1β (white bars), MCP-1 (gray bars), IL-6 (hatched bars) and IL-1RN (black bars) mRNA expression by GW3965.

FIG. 4 shows that LXR ligands inhibit iNOS and COX-2 gene expression by antagonizing NF-kB, a key inflammatory transcription factor. a and b, Expression and activation of LXRα/RXR (hatched bars) or LXRβ/RXR (black bars) inhibits induction of the iNOS (a) and COX-2 (b) promoters by LPS. c and d, Expression and activation of LXRα/RXR (hatched bars) or LXRβ/RXR (black bars) inhibits expression of a luciferase reporter containing multiple NF-κB binding sites (c), but not a reporter containing multiple AP-1 binding sites (d). e, A dominant-negative IKK expression vector (IKK-DN) blocks LXR-dependent repression of the COX-2 promoter. Transfected cells were treated with DMSO (white bars), GW3965 (gray bars), LPS (hatched bars), or GW3965 and LPS (black bars). f Expression and activation of LXRα/RXR (gray bars) fails to inhibit expression of COX-2 luciferase reporter lacking the NF-kB binding site. White bars—vector control (a-f).

FIG. 5 shows that LXR ligands reduce inflammation in vivo in a mouse model of cutaneous inflammation (irritant-induced contact dermatitis).

FIG. 6a shows that activation of LXR with LXR ligands inhibit the expression of NF-κB target genes iNOS and IL-6 in macrophage in response to TNFα and IL-1β. White bars—vehicle; hatched bars—T1317; black bars—GW3965. FIG. 6b shows that LXR ligand reduces the expression of the inflammatory molecule MMP-9 in atherosclerotic aortas of mice in vivo. White bars—vehicle; black bars—GW3965.

FIG. 7 shows that activation of LXR by synthetic ligands inhibits inflammatory gene expression in multiple human cell types. Two different synthetic LXR ligands, GW3965 (GW) and T1317 (T) inhibit TNF-induced gene expression in: A, human smooth muscle cells; B, human aortic endothelial cells; and C, human peripheral blood mononuclear cells (lymphocytes and monocytes).

FIG. 8 shows that the anti-inflammatory activity of LXR ligands can be separated from the induction of lipid metabolism genes such as ABCA1. A. Murine peritoneal macrophages were treated with LPS to induce iNOS gene expression. NOx production was measured by colorimetric assay. B. Murine peritoneal macrophages were assayed for ABCA1 mRNA by real time qPCR. In both experiments, cells were treated with increasing doses of two structurally different LXR ligands, T1317 and GW3965 as indicated.

### DETAILED DESCRIPTION OF THE PREFERRED EMBODIMENT

The present invention is a method for relieving inflammation and the symptoms of inflammation, including pain, fever, swelling, edema, and redness, that are associated with a variety of diseases and conditions, including, rheumatoid arthritis, juvenile rheumatoid arthritis, systemic lupus erythematosus, osteoarthritis, degenerative joint disease, one or more connective tissue diseases, ankylosing spondylitis, bursitis, Sjogren's syndrome, psoriasis, psoriatic arthritis, neuralgia, synoviitis, glomerulonephritis, vasculitis, sarcoidosis, atherosclerosis, asthma, inflammations that occur as sequellae to influenza, the common cold and other viral infections, gout, contact dermatitis, low back and neck pain, dysmenorrhea, headache, toothache, sprains, strains, myositis, burns, injuries, and pain and inflammation that follow surgical and dental procedures in a patient, where the method comprises the step of administering to the patient a therapeutically effective amount of an LXR agonist. In diseases that are auto-immune in nature, including, but not limited to, rheumatoid arthritis, juvenile rheumatoid arthritis, systemic lupus erythematosus, and many other degenerative joint diseases and connective tissue diseases, administration of an LXR agonist will, according to the instant invention, diminish the pathological inflammatory responses associated with these diseases. The invention is particularly useful in the treatment of rheumatoid arthritis.

Atherosclerosis is a disorder of lipid metabolism as well as a chronic inflammatory disease. Macrophages have a central role in atherogenesis through the accumulation of oxidized low-density lipoprotein (oxLDL) and the production of inflammatory mediators, cytokines and extracellular-matrix-degrading enzymes (Glass, C. K. & Witztum, J. L. 2001 Cell 104:503-516; Lusis, A. I. 2000 Nature 407:233-241). Mutations that alter lipid metabolic or immune functions of monocyte/macrophages have been shown to influence the development of atherosclerotic lesions in animal models. Inhibition of the ability of macrophages to accumulate lipid through genetic ablation of SR-A or CD36 reduces atherosclerosis (Febbraio, M. et al. 2000 J Clin Invest 105:1049-1056; Suzuki, H. et al. 1997 Nature 386:292-295). Mice lacking inducible nitric oxide synthase (iNOS), monocyte chemoattractant protein-1 (MCP-1), or bone marrow cyclooxygenase 2 (COX-2) expression also show a reduction in lesion formation on an atherosclerotic background (Detmers, P A et al. 2000 J Immunol 165:3430-3435; Gu, L. et al. 1998 Mol Cell 2:275-281; Kuhlencordt, P. I. et al. 2001 Circulation 103:3099-3104).

In addition to its well-established role as a substrate for macrophage lipid accumulation, oxLDL exerts complex effects on inflammatory gene expression. On one hand, oxLDL induces the expression of several inflammatory mediators in resting macrophages (Hansson, G. K. 1999 Curr Atheroscler Rep 1:150-155). On the other hand, oxLDL antagonizes certain inflammatory gene expression programs initiated after lipopolysaccharide (LPS) stimulation. For example, pretreatment with oxLDL has been shown to inhibit LPS-induced iNOS activity (Wu, Z. L. et al. 1998 Cell Biochem Funct 16:153-158). Biochemical studies have determined that oxidized cholesterol, rather than fatty acids or phosphatidylcholine, is responsible for this inhibition (Liu, S. X. et al. 1998 Atherosclerosis 136:43-49). The molecular mechanisms that mediate the effects of oxidized lipids on inflammatory gene expression are poorly understood.

Previous studies have suggested a role for peroxisome proliferator-activated receptor γ (PPARγ) in control of inflammation. Oxidized lipids derived from oxLDL activate PPARγ and induce expression of CD36 (Nagy, L. et al. 1998 Cell 93:229-240; Tontonoz, P. et al. 1998 Cell 93:241-252). Certain PPARγ ligands, especially 15-deoxy-Δ2,14-prostaglandin J2, antagonize the expression of iNOS, tumor necrosis factor (TNFα) and interleukin (IL)-6 in response to macrophage activation (Jiang, C. et al. 1998 Nature 391:82-86; Ricote, M. et al. 1998 Nature 391:79-82). Studies using PPARγ-deficient macrophages, however, have shown that at least some of these effects are independent of PPARγ (Chawla, A. et al. 2001 Nature Med 7:48-52; Moore, K. J. et al. 2001 Nature Med 7:41-47). Clearly, additional uncharacterized signaling pathways contribute to the regulation of inflammatory gene expression by oxidized lipids.

The liver X receptors (LXRα, encoded by the gene Nrlh3, and LXRβ, encoded by the gene Nrlh2) constitute a second class of nuclear receptors activated by oxidized lipids. LXRs are “cholesterol sensors” that regulate the expression of genes involved in lipid metabolism in response to specific oxysterol ligands (Repa, J. J. & Mangelsdorf, D. J. 2000 Annu Rev Cell Dev Biol 16:459-481). In macrophages, these oxysterols may be derived from internalized oxLDL or generated intracellularly through modification of cholesterol (Fu, X. et al. 2001 J Biol Chem 276:38378-38387; Laffitte, B. A. & Tontonoz, P. 2002 Curr Atheroscler Rep 4:213-221). Activation of LXR in macrophages induces expression of several genes involved in lipid metabolism and reverse cholesterol transport, including Abca1, Abcg1 and Apoe (Repa, J. J. & Mangelsdorf, D. J. 2002 Nature Med 8:1243-1248). Recent studies have demonstrated that LXRs exert an important atheroprotective effect in macrophages. Systemic administration of an LXR agonist reduced atherosclerosis in Ldlr^{-/-} and Apoe^{-/-} mice (Joseph, S. B. et al. 2002 PNAS USA 99:7604-7609). Conversely, loss of LXR expression from bone marrow increases lesion formation in these same models (Tangirala, R. K. et al. 2002 PNAS USA 99:11896-11901). However, though the liver X receptors (LXRs) are established mediators of lipid-inducible gene expression, their role in inflammation and immunity is unknown.

The data presented herein demonstrate that LXR agonists have utility for treating, preventing, controlling or reducing the risk of contracting one or more diseases or conditions selected from rheumatoid arthritis, juvenile rheumatoid arthritis, systemic lupus erythematosus, osteoarthritis, degenerative joint disease, one or more connective tissue diseases, ankylosing spondylitis, bursitis, Sjogren's syndrome, psoriasis, psoriatic arthritis, neuralgia, synoviitis, glomerulonephritis, vasculitis, sarcoidosis, atherosclerosis, asthma, inflammations that occur as sequellae to influenza, the common cold and other viral infections, gout, contact dermatitis, low back and neck pain, dysmenorrhea, headache, toothache, sprains, strains, myositis, burns, injuries, and pain and inflammation that follow surgical and dental procedures in a patient, where the method of treatment comprises the steps of first identifying a mammalian patient in need of treatment, including preventive treatment, and then administering a therapeutically effective amount of an LXR agonist, or a pharmaceutically acceptable salt thereof, to the patient. The patient is a mammal, and preferably is a human.

In preferred embodiments, the LXR compounds may be used to treat, prevent, control or reduce the risk of contracting one or more auto-immune diseases or disorders selected from systemic lupus erythematosus, rheumatoid arthritis, juvenile rheumatoid arthritis, glomerulonephritis, nephritis, vasculitis, sarcoidosis, degenerative joint disease, Sjogren's syndrome, psoriasis, psoriatic arthritis, dysmenorrhea, myositis, neuralgia, synoviitis, ankylosing spondylitis and bursitis, by the steps of first identifying a mammalian patient in need of treatment, including preventive treatment, and then administering a therapeutically effective amount of an LXR agonist, or a pharmaceutically acceptable salt thereof, to the patient.

In a preferred embodiment, the LXR agonist may be used to treat, prevent, control, or reduce the risk of contracting rheumatoid arthritis, juvenile rheumatoid arthritis, systemic lupus erythematosus, osteoarthritis, degenerative joint disease, one or more connective tissue diseases, ankylosing spondylitis, and bursitis, by the steps of first identifying a mammalian patient in need of treatment, including preventive treatment, and then administering a therapeutically effective amount of an LXR agonist, or a pharmaceutically acceptable salt thereof, to the patient.

In a preferred embodiment, the LXR agonist may be used to treat, prevent, control, or reduce the risk of contracting rheumatoid arthritis, juvenile rheumatoid arthritis, systemic lupus erythematosus, osteoarthritis, degenerative joint disease, and one or more connective tissue diseases, by the steps of first identifying a mammalian patient in need of treatment, including preventive, treatment, and then administering a therapeutically effective amount of an LXR agonist, or a pharmaceutically acceptable salt thereof, to the patient.

In particularly preferred embodiments, the LXR compounds may be used to treat, prevent, control or reduce the risk of contracting rheumatoid arthritis, or juvenile rheumatoid arthritis by the steps of first identifying a mammalian patient in need of treatment, including preventive treatment, and then administering a therapeutically effective amount of an LXR agonist, or a pharmaceutically acceptable salt thereof, to the patient.

In another particularly preferred embodiments, the LXR compounds may be used to treat, prevent, control or reduce the risk of contracting osteoarthritis or degenerative joint disease by the steps of first identifying a mammalian patient in need of treatment, including preventive treatment, and then administering a therapeutically effective amount of a LXR agonist, or a pharmaceutically acceptable salt thereof, or a prodrug thereof, to the patient.

“LXR” means nuclear receptors LXRα and LXRβ and all subtypes, as well as the corresponding genes. LXRβ includes human LXRβ (GenBank Accession Number P55055; Apfel, et al. 1994 Mol Cell Biol 14:7025-7035; Willy, et al. 1995 Genes Dev 9:1033-1045; and Long, et al. 1995 PNAS USA 91:10809-10813). LXRβ includes LXRb, LXR beta, NER, NER1, UR, OR-1, R1P15, NR1H2 or any homologue.

Some LXR agonists are known in the art. For example, see U.S. 2003/0073614, Collins et al. 2002 J Med Chem 45:1963-1966, and Repa, J. J. et al. 2000 Science 289:1524-1529.

In preferred embodiments of the present invention, the LXR receptor agonists are selected from the group consisting of GW3965 (Collins J. L. et al. 2002 J Med Chem 45:1963-1966) and T0901317 (Schultz et al. 2000 Genes Dev 14:2831-2838). These LXR agonists are proprietary compounds owned by GlaxoSmithKline.

GW3965 has the formula:

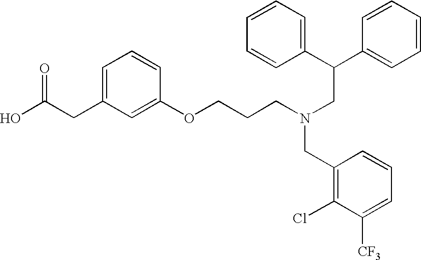

T0901317 has the formula: (N-(2,2,2-trifluoro-ethyl)-N-[4-(2,2,2-trifluoro-1-hydroxy-1-trifluoromethyl-ethyl)-phenyl]-benzenesulfonamide):

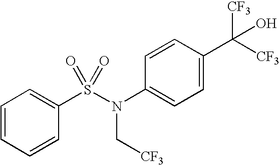

Dosage and Administration. The LXR agonists of the invention can be administered in such oral forms as tablets, capsules (each of which includes sustained release or timed release formulations), pills, powders, granules, elixers, tinctures, suspensions, syrups, and emulsions. Likewise, they may be administered in intravenous (bolus or infusion), intraperitoneal, subcutaneous, topical, or intramuscular form, all using forms well known to those of ordinary skill in the pharmaceutical arts.

The LXR agonists can be administered in the form of a depot injection or implant preparation which may be formulated in such a manner as to permit a sustained release of the active ingredient. The active ingredient can be, compressed into pellets or small cylinders and implanted subcutaneously or intramuscularly as depot injections or implants. Implants may employ inert materials such as biodegradable polymers or synthetic silicones, for example, Silastic, silicone rubber or other polymers manufactured by the Dow-Corning Corporation.

The LXR agonists can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The LXR agonists may also be delivered by the use of monoclonal antibodies as individual carriers to which the compound molecules are coupled. The LXR agonists may also be coupled with soluble polymers as targetable drug carriers. Such polymers can include, but are not limited to, polyvinlypyrrolidinonepyran copolymer, poly(hydroxypropyl)methacrylamide-phenol copolymer, polyhydroxyethylaspartamide-phenol copolymer, or polyethyleneoxide-polylysine copolymer substituted with palmitoyl residues. Furthermore, the LXR agonists may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross-linked or amphipathic block copolymers of hydrogels.

The dosage regimen utilizing the LXR agonists is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, alleviate, control or stop the symptoms and the progress of the condition.

Oral administration is the preferred route of drug delivery when oral administration is practicable. Oral dosages of the LXR agonists, when used for the indicated effects, will generally range between about 0.001 mg per kg of body weight per day (mg/kg/day) to about 100 mg/kg/day, and preferably about 0.10 mg/kg/day (unless specified otherwise, amounts of active ingredients are on the basis of a neutral molecule, which may be a free acid or free base). For example, an 80 kg patient would receive between about 0.08 mg/day and 8 g/day, and preferably between about 0.8 mg/day and 800 mg/day. A suitably prepared medicament for once a day administration would thus contain between 0.08 mg and 8 g, and preferably between 0.8 mg and 800 mg. Advantageously, the LXR agonists may be administered in divided doses of two, three, or four times daily. For administration twice a day, a suitably prepared medicament as described above would contain between 0.04 mg and 4 g, and preferably between 0.4 mg and 400 mg. Dosages outside of the aforementioned ranges may be necessary in some cases. Carrier would generally be added in an amount that would constitute about 5%-95% of the total composition. Examples of daily dosages that may be given in the range of 0.08 mg-8 g per day include 0.1 mg, 0.5 mg, 1 mg, 5 mg, 10 mg, 25 mg, 50 mg, 100 mg, 200 mg, 300 mg, 400 mg, 500 mg, 600 mg, 800 mg, 1 g, 2 g, 4 g and 8 g. These would be divided into smaller doses if administered more than once per day (e.g., one-half the amount in each administration if the drug is taken twice daily).

Intravenously or subcutaneously, the patient would receive the injected doses in a quantity that would deliver the active ingredient in approximately the quantities described above. The quantities may be adjusted to account for differences in the efficiency of delivery that results from the use of a mode of delivery that bypasses the digestive system. Such quantities may be administered in a number of suitable ways, e.g. large volumes of low concentrations of active ingredient during one extended period of time or several times a day, low volumes of high concentrations of active ingredient during a short period of time, e.g., once a day.

Typically, a conventional intravenous formulation may be prepared which contains a concentration of active ingredient of between about 0.1-1.0 mg/ml, such as for example 0.1 mg/ml, 0.3 mg/ml, or 0.6 mg/ml, and administered in amounts per day equivalent to the amounts per day stated above. For example, an 80 kg patient, receiving 8 ml twice a day of an intravenous formulation having a concentration of active ingredient of 0.5 mg/ml, receives 8 mg of active ingredient per day.

Glucuronic acid, L-lactic acid, acetic acid, citric acid or any pharmaceutically acceptable acid/conjugate base with reasonable buffering capacity in the pH range acceptable for intravenous administration may be used as buffers. Consideration should be given to the solubility and chemical compatibility of the drug in choosing an appropriate excipient. Subcutaneous formulations, preferably prepared according to procedures well known in the art at a pH in the range between 7.0 and 7.4, also include suitable buffers and isotonicity agents. They are formulated to deliver a daily dose of LXR agonist in one or more daily subcutaneous administrations, e.g., one, two or three times each day. The choice of appropriate buffer and pH of a formulation, depending on solubility of the drug to be administered, is readily made by a person having ordinary skill in the art.

The compounds can also be administered in intranasal form via topical use of suitable intranasal vehicles as a spray or inhaler, or via transdermal routes, using those forms of transdermal skin patches or creams well known to those of ordinary skill in that art. To be administered in the form of a transdermal delivery system, the dosage administration will, or course, be continuous rather than intermittent throughout the dosage regime.

The LXR agonists are typically administered as active ingredients in admixture with suitable pharmaceutical diluents, excipients or carriers (collectively referred to herein as “carrier” materials) suitably selected with respect to the intended form of administration, that is, oral tablets, capsules, elixers, syrups and the like, and consistent with convention pharmaceutical practices.

For instance, for oral administration in the form of a tablet or hard capsule, the active drug component can be combined with an oral, non-toxic, pharmaceutically acceptable, inert carrier such as lactose, starch, sucrose, glucose, methyl cellulose, magnesium stearate, dicalcium, phosphate, calcium sulfate, mannitol, sorbitol and the like. For oral administration in liquid form, the oral drug components can be combined with any oral, non-toxic, pharmaceutically acceptable inert carrier such as ethanol, glycerol, water and the like. The oral dosage form may also be suspended in an oil, such as a vegetable oil, which could be, without limitation, arachis oil, olive oil, sesame oil or coconut oil, or a mineral oil, such as paraffin oil. Moreover, when desired or necessary, suitable binders, lubricants, disintegrating agents and coloring agents can also be incorporated into the mixture.

Suitable binders include starch, gelatin, natural sugars such as glucose or beta-lactose, corn-sweeteners, natural and synthetic gums such as acacia, tragacanth or sodium alginate, carboxymethylcellulose, polyethylene glycol, waxes and the like.

Lubricants used in these dosage forms include sodium oleate, sodium stearate, magnesium stearate, sodium benzoate, sodium acetate, sodium chloride and the like.

Disintegrators include, without limitation, starch, methyl cellulose, agar, bentonite, xanthan gum and the like.

### Definitions

“Pharmaceutically acceptable salts” means non-toxic salts of the compounds employed in this invention which are generally prepared by reacting the free acid with a suitable organic or inorganic base. Examples of salt forms of LXR agonists may include, but are not limited to, acetate, benzenesulfonate, benzoate, bicarbonate, bisulfate, bitartrate, borate, bromide, calcium, calcium edetate, camsylate, carbonate, chloride, clavulanate, citrate, dihydrochloride, edetate, edisylate, estolate, esylate, fumarate, gluceptate, gluconate, glutamate, hexylresoreinate, hydrabamine, hydrobromide, hydrochloride, hydroxynapthoate, isothionate, lactate, lactobionate, laurate, malate, maleate, mandelate, mesylate, methylsulfate, mucate, oleate, oxalate, pamaote, palmitate, panthothenate, phosphate/diphosphate, polygalacturonate, potassium, salicylate, sodium, stearate, subacetate, succinate, tannate, tartrate, teoclate, tosylate, and valerate. Examples of salt forms of COX-2 inhibitors include but are not limited to salts derived from inorganic bases including aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaniinoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Unless defined otherwise, “therapeutically effective amount” means that amount of a drug or pharmaceutical agent that will elicit the biological or medical response of a tissue, a system, animal or human that is being sought by a researcher, veterinarian, medical doctor or other clinician.

Unless defined otherwise, “prophylactically effective amount” means that amount of a pharmaceutical drug that will prevent or reduce the risk of occurrence of the biological or medical event that is sought to be prevented in a tissue, a system, animal or human by a researcher, veterinarian, medical doctor or other clinician.

Combination Therapy. Similarly, LXR agonists may be useful as a partial or complete substitute for conventional NSAID's in preparations where NSAID's are presently coadministered with other agents or ingredients. Thus in further aspects, the invention encompasses pharmaceutical compositions for treating inflammatory diseases as defined above comprising a non-toxic therapeutically effective amount of an LXR agonist as defined above and one or more ingredients such as another pain reliever; an NSAID; a potentiator including caffeine; an H2-antagonist; aluminum or magnesium hydroxide; simethicone; a decongestant; an antitussive; a diuretic; and a sedating or non-sedating antihistamine. In addition the invention encompasses a method of treating inflammatory diseases comprising administration to a patient in need of such treatment a non-toxic therapeutically effect amount of an LXR agonist, optionally co-administered with one or more of such ingredients as listed immediately above.

Examples of antitussives include codeine, hydrocodone, caramiphen, carbetapentane, and dextramethorphan.

Examples of decongestants include phenylephrine, phenylpropanolamine, pseudoephedrine, oxymetazoline, ephinephrine, naphazoline, xylometazoline, propylhexedrine, or levo-desoxyephedrine.

Examples of pain relievers include acetominophen and phenacetin.

Combination of LXR Agonist and NSAID or Corticosteroid. The instant invention also involves a novel combination therapy comprising the administration of a therapeutically effective amount of an NSAID such as a selective or non-selective COX-2 inhibitor or corticosteroid in combination with a therapeutically effective amount of an LXR agonist to a mammal, and more particularly, to a human. The combination therapy is used to treat inflammation and inflammatory diseases.

The pharmaceutical combinations comprising an LXR agonist in combination with an NSAID such as a COX-2 inhibitor or corticosteroid include single pharmaceutical dosage formulations which contain both the LXR agonist and the NSAID or corticosteroid in a single dose, as well as formulations in which each active agent is administered in its own separate pharmaceutical dosage formulation. Where separate dosage formulations are used, the LXR agonist and the NSAID or the corticosteroid can be administered at essentially the same time, i.e., concurrently, or at separately staggered times, i.e., sequentially. The “instant pharmaceutical combination” is understood to include all these regimens. Administration in these various ways are suitable for the present invention as long as the beneficial pharmaceutical effect of the LXR agonist and the NSAID or the LXR agonist and the corticosteroid are realized by the patient at substantially the same time. Such beneficial effect is preferably achieved when the target blood level concentrations of each active drug are maintained at substantially the same time. It is preferred that the LXR agonist and the NSAID or corticosteroid be co-administered concurrently on a once-a-day dosing schedule; however, varying dosing schedules, such as the LXR agonist once per day and the NSAID or corticosteroid once, twice or more than twice per day, or the NSAID or corticosteroid once per day and the LXR agonist once, twice or more than twice per day, is also encompassed herein. A single oral dosage formulation comprised of both the LXR agonist and the NSAID is preferred. A single dosage formulation will provide convenience for the patient.

The instant invention also provides pharmaceutical compositions comprised of a therapeutically effective amount of an NSAID, or a pharmaceutically acceptable salt thereof, or a corticosteroid in combination with a therapeutically effective amount of an LXR agonist, or a pharmaceutically acceptable salt thereof, and a pharmaceutically acceptable carrier. One embodiment of the instant compositions is a single composition adapted for oral administration comprised of a therapeutically effective amount of a COX-2 inhibitor in combination with a therapeutically effective amount of an LXR agonist and a pharmaceutically acceptable carrier. The combination can also be administered in separate dosage forms, each having one of the active agents. If administered in separate dosage forms, the separate dosage forms are administered such that the beneficial effect of each active agent is realized by the patient at substantially the same time.

Common NSAIDs include salicylates such as aspirin, sodium salicylate, choline salicylate, salicylsalicylic acid, diflunisal, and salsalate; indoleacetic acids such as indomethacin and sulindac; pyrazoles such as phenylbutazone, oxyphenbutazone; pyrrolealkanoic acids such as tolmetin; phenylacetic acids such as ibuprofen, feroprofen, flurbiprofen, and ketoprofen; fenamates such as mefanamic acid, and meclofenamate; oxicams such as piroxicam; and naphthaleneacetic acids such as naproxen. These are all non-selective NSAID's, inhibiting both cyclooxygenase-1 (COX-1) and cyclooxygenase-2 (COX-2) receptors.

Selective cyclooxygenase-2 (COX-2) inhibitors are particularly advantageous for patients who are sensitive to the side effects of cyclooxygenase-1 (COX-1) inhibition that results when non-selective NSAID's are administered. Examples of COX-2 inhibitors that may be advantageously used in combination therapy with LXR agonists include rofecoxib, etoricoxib, celecoxib, parecoxib and valdecoxib.

Common corticosteroid used for the treatment of inflammation include hydrocortisone, prednisolone, 6-alpha-methylprednisolone, triamcinolone, dexamethasone and betaroethasone.

The dosage regimen utilizing a LXR agonist in combination with the NSAID or with the corticosteroid is selected in accordance with a variety of factors including type, species, age, weight, sex and medical condition of the patient; the severity of the condition to be treated; the route of administration; the renal and hepatic function of the patient; and the particular compound or salt or ester thereof employed. Since two different active agents are being used together in a combination therapy, the potency of each of the agents and the interactive effects achieved by combining them together must also be taken into account. A consideration of these factors is well within the purview of the ordinarily skilled clinician for the purpose of determining the therapeutically effective or prophylactically effective dosage amounts needed to prevent, counter, or arrest the progress of the condition.

Administration of the drug combination to the patient includes both self-administration and administration to the patient by another person.

Additional active agents may be used in combination with the NSAID and LXR agonist or in combination with an LXR agonist and a corticosteroid in a single dosage formulation, or may be administered to the patient in a separate dosage formulation, which allows for concurrent or sequential administration. Examples of additional active agents which may be employed include HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors; probucol; niacin; PPAR-α agonists, including fibrates such as clofibrate, fenofibrate, and gemfibrizol; PPAR-γ agonists and PPARα/γ dual agonists, which are insulin sensitizers; cholesterol absorption inhibitors; bile acid sequestrants; LDL (low density lipoprotein) receptor inducers; vitamin B6 (also known as pyridoxine) and the pharmaceutically acceptable salts thereof such as the HCl salt; vitamin B₁₂ (also known as cyanocobalamin); β-adrenergic receptor blockers; folic acid or a pharmaceutically acceptable salt or ester thereof such as the sodium salt and the methylglucamine salt; and anti-oxidant vitamins such as vitamin C and E and beta carotene.

An additional embodiment of the instant invention involves a kit comprising an NSAID such as a COX-2 inhibitor in an oral dosage formulation and an LXR agonist in a separate oral dosage formulation.

Another embodiment of the instant invention involves a kit comprising an anti-inflammatory corticosteroid in an oral dosage formulation and an LXR agonist in a separate dosage formulation.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be not limited by the scope of the following examples.

### EXAMPLES

To determine the molecular basis for the atheroprotective effects of liver X receptors (LXRs), other roles for these receptors in macrophage biology were explored. Surprisingly, a previously unrecognized role for LXRs in the control of inflammatory gene expression was discovered. Indeed, the LXR pathway was identified as a common regulator of lipid metabolic and immune functions in macrophages, indicating that LXR ligands have utility in macrophage-dependent inflammatory diseases. Such diseases include but not limited to atherosclerosis and rheumatoid arthritis, as well as glomerulonephritis.

Inhibition of LPS-Induced iNOS Production by LXR Agonists

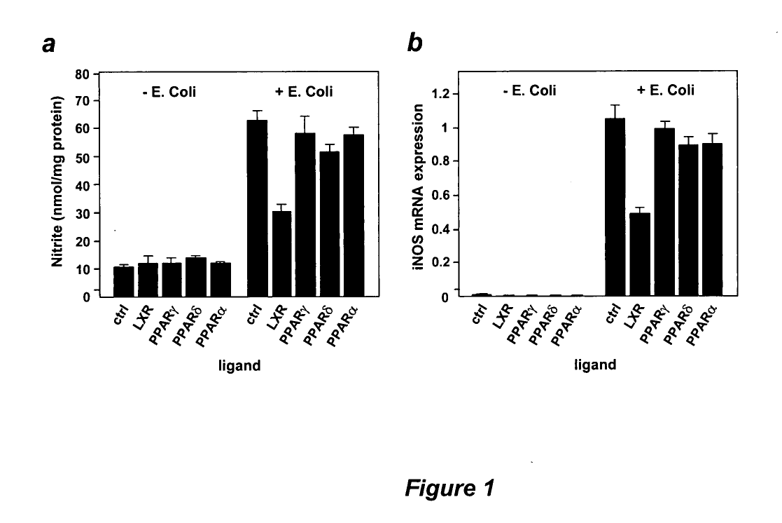

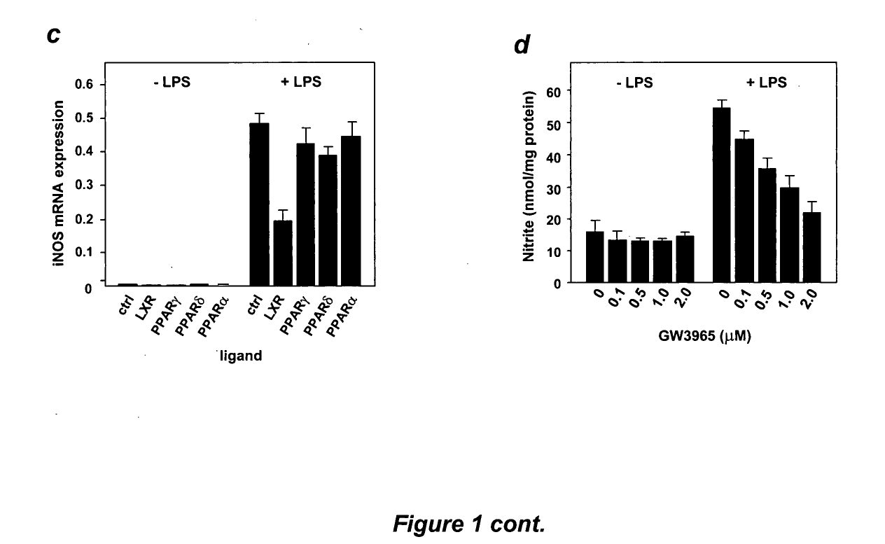

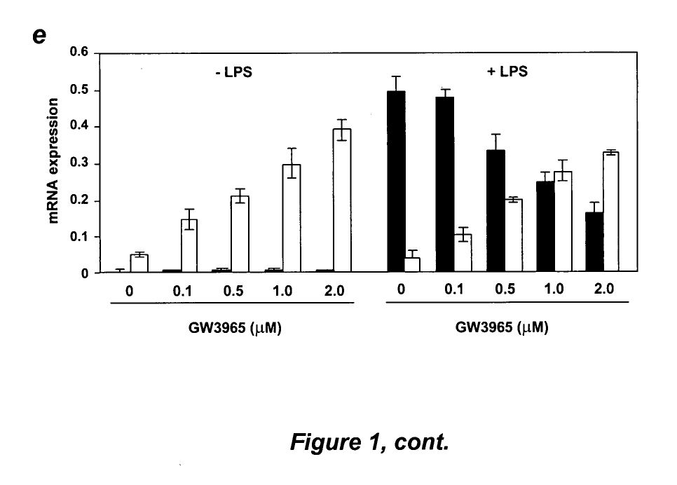

To investigate the role of LXRs in host defense, the influence of LXR agonists on macrophage responses to bacterial pathogens was tested. Thioglycolate-elicited peritoneal macrophages were infected in vitro with Escherichia coli. After 18 hours, expression of inducible nitric oxide synthase (iNOS) mRNA and nitric oxide (NO) production were assessed. Macrophages pretreated with the LXR agonist GW3965 showed significantly reduced iNOS mRNA and reduced iNOS activity compared with those treated with vehicle alone (FIGS. 1A and B). This repression occurs over the same concentration range needed to induce the established LXR target gene ATP-binding cassette transporter A1 (ABCA1). A, LXR agonist inhibits nitrite production in response to bacterial infection. B, LXR agonist inhibits induction of iNOS mRNA in response to bacterial infection. C, LXR agonist inhibits induction of iNOS mRNA in response to LPS. D, Dose-dependent inhibition of nitrite production by GW396S. E, Reciprocal dose-dependent regulation of ABCA1 (black bars) and iNOS (gray bars) mRNA expression by GW3965. Thioglycolate-elicited murine peritoneal macrophages were pretreated with vehicle or 1 μM GW3965 (LXR), rosiglitazone (peroxisome proliferator-activated receptor γ, PPARγ), GW1516 (PPARδ, PNAS 98:5306) or GW7647 (PPARα, PNAS 98:5306). Cells were then infected with E. coli (A, B) or treated with 100 ng/ml LPS(C, D, E) as indicated. After 18 h, NO production was determined using a colorimetric assay (A, D), or mRNA levels were determined using real-time quantitative PCR assays (B, C, E). A similar inhibition was observed in response to stimulation with lipopolysaccharide (LPS) (100 ng/ml; FIG. 1E). The inhibitory effect of LXR agonists on iNOS expression was dose-dependent and occurred over the same range of concentration as that required to induce ATP-binding cassette transporter A1 (ABCA1) (FIGS. 1D and E). In contrast, ligands for peroxisome proliferator-activated receptor γ (PPARγ) (rosiglitazone), PPARδ (GW1516) and PPARα (GW7647) had minimal effects on iNOS expression at concentrations of 1 μM (FIG. 1A-C).

Reciprocal Regulation of Genes Involved in Lipid Metabolism and Inflammation by LXRs

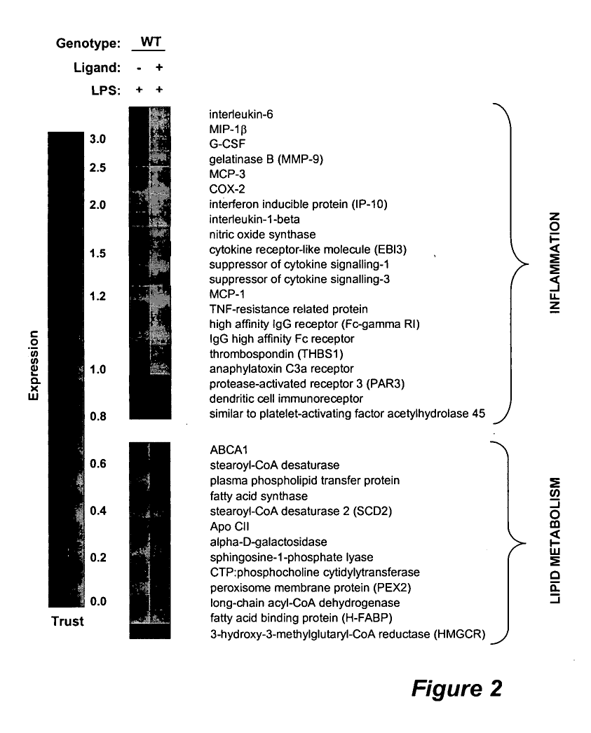

DNA microarrays were used to further examine the effect of LXR activation on LPS-induced gene transcription. Thioglycolate-elicited peritoneal macrophages from wild-type mice were treated with 2 μM GW3965 for 18 hours before treatment with 100 ng/ml LPS. Cells were harvested 6 hours after LPS stimulation and total RNA from these samples was used to probe Affymetrix U74Av2 arrays. The array data were analyzed using GeneSpring software to identify genes regulated by the ligand in the cells. Selected genes fitting these criteria are represented graphically in FIG. 2. Graphic representation of gene expression changes in macrophages treated with 100 ng/ml LPS and vehicle or synthetic LXR agonist GW3965 (2 μM) as indicated. Genes involved in inflammation or lipid metabolism were selected based on ligand-dependent changes in expression in the cells. The most highly induced genes were those involved in lipid metabolism, including established LXR target genes such as Abca 1. A large cluster of genes involved in the macrophage innate immune response was inhibited by the LXR agonist. These genes encoded iNOS and COX-2, cytokines such as IL-6, IL-1β and granulocyte colony-stimulating factor (G-CSF), chemokines such as monocyte chemoattractant protein-1 (MCP-1), MCP-3, macrophage inflammatory protein-1β (MIP-1β) and interferon-inducible protein-10 (IP-10), and the metalloproteinase MMP-9. Array analysis also confirmed the specificity of GW3965 and showed that the expression of genes involved in other cellular processes was not significantly altered by LXR ligand (see Table 1). Thus, in activated macrophages, the cholesterol efflux pathway and the innate immune response are reciprocally regulated by LXRs (Table 1).

TABLE 1
Genes not significantly altered by LXR ligand GW3965.

| Gene name | Change |
|---|---|
| cell-cycle | |
| Cyclin-dependent kinase inhibitor 1C (P57) | 1.28 |
| Growth arrest specific 7 | 1.22 |
| Avian reticuloendotheliosis viral (v-rel) oncogene related B | 1.21 |
| Cyclin dependent kinase inhibitor 2B (Cdkn2b) | 1.15 |
| Proliferating cell nuclear antigen | 1.13 |
| Met proto-oncogene | 1.12 |
| Guanine Nucleotide Regulatory Protein | 1.12 |
| Ki-ras cellular oncogene exon 1 from y1 adrenal tumor cells | 1.10 |
| Cell division cycle 42 | 1.06 |
| Cyclin 1 | 1.05 |
| Cell cycle checkpoint protein | 1.04 |
| Transcription factor junB (junB) gene | 1.04 |
| Cyclin-dependent kinase 5 | 1.00 |
| Early growth response 2 | 0.96 |
| Growth arrest specific 2 | 0.92 |
| Growth arrest specific 1 | 0.91 |
| Cyclin-dependent kinase inhibitor 1A (P21) | 0.85 |
| Cyclin G2 | 0.83 |
| Cyclin F | 0.72 |
| Cyclin B2 | 0.68 |
| apoptosis | |
| Caspase 3, apoptosis related cysteine protease | 1.40 |
| Bc12-associated X protein | 1.15 |
| E1B 19 K/Bc1-2-binding protein homolog (Nip3) | 1.14 |
| Bc1-10 protein | 1.13 |
| Caspase 2 | 1.10 |
| Apoptosis inhibitor 2 | 1.10 |
| Apoptosis signal-regulating kinase 1 | 0.99 |
| Bc12 homologous antagonist/killer | 0.94 |
| Caspase 7 | 0.94 |
| Caspase 11 | 0.92 |
| Apoptosis inhibitor 3 | 0.83 |
| Apoptosis-inducing factor AIF (Aif) | 0.83 |
| Bc12-like | 0.80 |
| Caspase 9 | 0.79 |

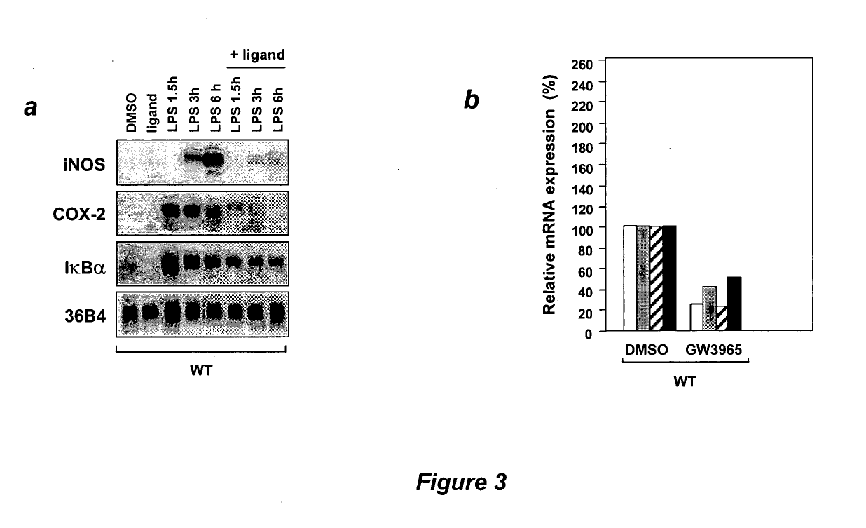

LPS is an established inducer of the NF-κB signaling pathway in macrophages. Several genes inhibited by LXR ligands in the array studies above are established targets of NF-κB. To further investigate the role of LXRs in NF-κB-dependent gene expression, we analyzed expression of NF-κB target genes in peritoneal macrophages from wild-type mice. Thioglycolate-elicited murine peritoneal macrophages from wild-type mice were pretreated for 18 h with GW3965 before the addition of 100 ng/ml LPS. Expression of NF-κB target genes was determined by northern blot (A) or by real-time quantitative PCR assays (B). 36B4 was used as a control for RNA loading and integrity. Northern blot analysis (3 hours and 6 hours after LPS stimulation, FIG. 3A) and real-time quantitative PCR analysis (6 hours after LPS stimulation, FIG. 3B) confirmed that LXR agonists were effective inhibitors of COX-2, lκBα, IL-1β, MCP-1, IL-6 and IL1-receptor antagonist (IL-1RN) expression (FIGS. 3A and B). A modest receptor-independent induction of MCP-1 and IL-6 was observed in response to both GW3965 and T01317.

Activation of LXR Antagonizes NF-κB Signaling

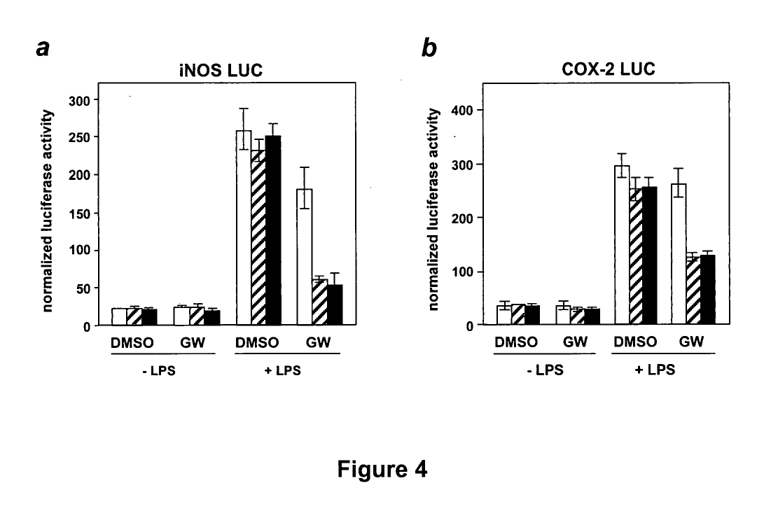

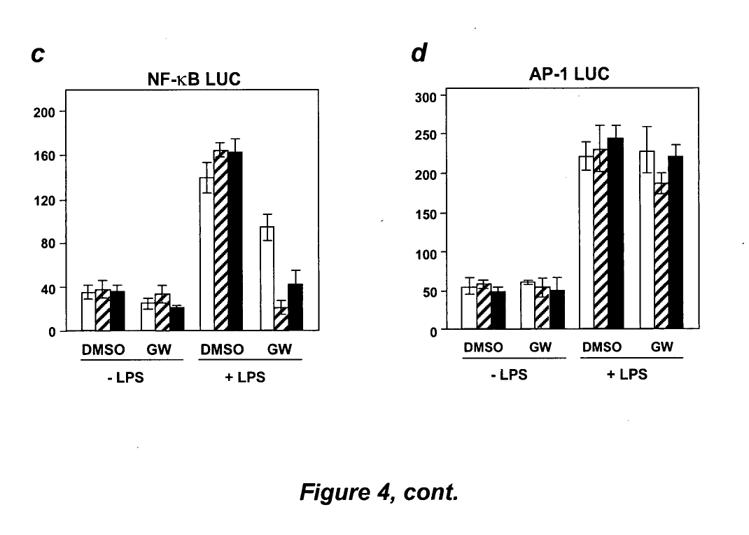

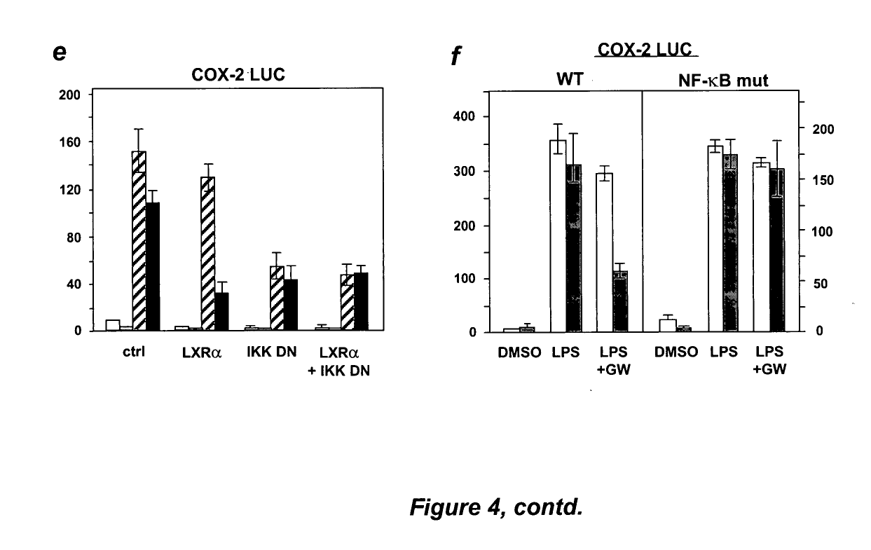

We next investigated the mechanism by which LXR inhibits iNOS and COX-2 expression. The iNOS and COX-2 promoters contain binding sites for NF-κB that are required for maximal responses to LPS (Lowenstein, C J. et al. 1993 PNAS USA 90:9730-9734; Mestre, I. R. et al. 2001 J Biol Chem 276:977-3982), but do not contain LXR binding sites. RAW 264.7 cells were transfected with expression vectors for either LXRα/RXRα or LXRβ/RXRα along with luciferase reporter constructs. The LXR agonist inhibited expression of both iNOS and COX-2 gene promoters in a receptor-dependent fashion (FIG. 4 A and B). Expression and activation of LXRα/RXR or LXRβ/RXR inhibits induction of the iNOS (A) and COX-2 (B) promoters by LPS. C and D, Expression and activation of LXRα/RXR or LXRβ/RXR inhibits expression of a luciferase reporter containing multiple NF-κB binding sites (C), but not a reporter containing multiple AP-1 binding sites (D). E, A dominant-negative IKK expression vector (IKK DN) blocks LXR-dependent repression of the COX-2 promoter. Transfected cells were treated with DMSO (ctrl), GW3965 (GW), LPS, or GW3965 and LPS. F, Expression and activation of LXRα/RXR (gray bars) fails to inhibit expression of a COX-2 luciferase reporter lacking the NF-κB binding site. RAW 264.7 cells were transiently transfected with expression vectors for LXRα/RXRα, LXRβ/RXRα or dominant-negative I kappa B kinase (IKK) along with iNOS (A), COX-2 (B, E, F), NF-κB (C), AP-1 (D) or COX-2/NF-κB mutant (F) luciferase reporters. Following transfection, cells were treated with vehicle or 2 μM GW3965 (GW) before the addition of LPS, as indicated. As activation of iNOS and COX-2 gene promoters by LPS is known to depend on interactions between AP-1 and NF-κB transcription factors, the effect of LXR on minimal promoters containing binding sites for these factors was evaluated. LXR ligand effectively inhibited the reporter containing binding sites for NF-κB, but not that containing binding sites for AP-1 (FIGS. 4C and D). Moreover, the inhibitory effect of LXR on the COX-2 promoter was lost when NF-κB activity was blocked by transfection of a dominant-negative IKK or when binding sites for NF-κB were mutated (FIGS. 4E and F). These observations indicate that inhibition of inflammatory gene expression by LXR ligands involves antagonism of NF-κB signaling.

LXRs Have Anti-Inflammatory Activity In Vivo

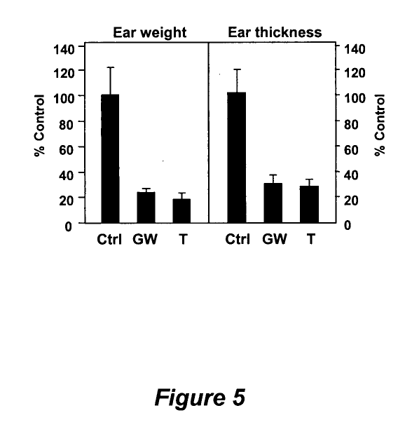

The effects of LXR ligands in both acute and chronic inflammatory contexts were evaluated. First, the effects of LXR agonists in a murine model of irritant contact dermatitis (Sheu, M. Y. et al. 2002 J Invest Dermatol 118:94-101) were studied. Ears of C57B1/6 mice (n=5 per group) were treated topically with 12-O-tetradecanoylphorbol-13-acetate (TPA) (left ear) or with vehicle (right ear). Vehicle (ctrl), GW3965 (GW) or T1317 (T) was applied topically post-TPA treatment. Thickness and weight of 6-mm punch biopsies were determined for each group after 18 h TPA treatment and are presented relative to vehicle-treated ear. Ear thickness and weight were increased following TPA treatment, and both parameters were reduced by treatment with GW3965 or T1317 (FIG. 5). Analysis of hematoxylin and eosin (H&E) stained sections of the TPA-treated ears confirmed a reduction in edema and inflammatory infiltrate by LXR agonist. Thus, two structurally unrelated LXR agonists have anti-inflammatory activity in a model of cutaneous inflammation.

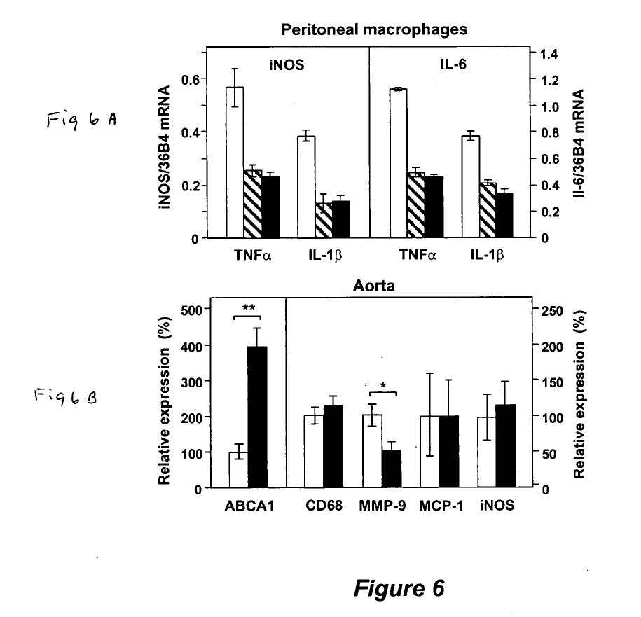

Next, the influence of LXR agonists on inflammatory gene expression induced by IL-1β and TNFα, which have been implicated in the pathogenesis of atherosclerosis, was tested. As observed above for LPS, GW3965 inhibited the induction of iNOS and IL-6 expression by IL-1β or TNFα (50 ng/ml). Finally, real-time quantitative PCR was used to analyze gene expression in the atherosclerotic aortas of Apoe^{-/-} mice treated with vehicle or GW3965 for 3 days. FIG. 6A, Thioglycolate-elicited murine peritoneal macrophages were pretreated for 18 h with T1317 (hatched bar) or GW3965 (black bar) before the addition of IL-1 or TNFα (50 ng/ml). Gene expression was detected by real-time quantitative PCR assays. FIG. 6B, Apoe^{-/-} mice of 8 months of age (n=5 per group) were treated with vehicle (white bars) or GW3965 (black bars) for 3 days. Total RNA was isolated from individual aortas and gene expression was measured by real-time quantitative PCR. P<0.05 for MMP-9 (*) and P<0.01 (**) for ABCA1. The LXR ligand induced expression of ABCA1, but did not affect expression of the macrophage-specific marker CD68, indicating that the number of macrophages present was similar in each group (FIG. 6B). Expression of MCP-1 and iNOS was not different between the two groups; however, expression of MMP-9 was substantially reduced in mice treated with LXR agonist. Thus, LXR agonist reduces expression of an established inflammatory mediator in the vessel wall.

Macrophages have a central role in innate immunity. They function to scavenge pathogens and apoptotic cells as well as to coordinate the inflammatory response to such stimuli through the production of cytokines and other mediators. Previous studies have suggested that LXRs way play role in the macrophage cholesterol efflux pathway. Here, the possibility that cellular lipid accumulation might affect the production of inflammatory mediators by activating LXR was explored. Global analysis of gene expression in activated macrophages revealed that LXRs inhibit genes involved in the innate immune response while simultaneously inducing those involved in lipid metabolism. In vivo, activation of the LXR pathway was shown to antagonize inflammatory gene expression and to reduce inflammation in a model of contact dermatitis. These observations identify LXR as a molecular link between lipid metabolism and inflammation.

The function of LXRs as cholesterol sensors in macrophages is envisioned to be involved in the scavenger function of these cells. Internalization of apoptotic cells and cellular debris leads to the accumulation of large amounts of fatty acid and cholesterol. In this context, the role of LXR is to activate the cholesterol efflux pathway to protect the cell from lipid overload. The present results indicate for the first time that another consequence of LXR activation in cholesterol-loaded cells is to limit the production of inflammatory mediators. When apoptotic cells are being scavenged, an inflammatory response is not appropriate. In fact, the absence of inflammation is a hallmark of apoptotic cell death. In contrast, phagocytosis of pathogens, such as Gram-negative bacteria, elicits a marked immune response. Not only do pathogens contain molecules such as LPS that are recognized by the Toll family of receptors, they also lack cholesterol and therefore would not be expected to activate the LXR pathway. Thus, LXRs are envisioned to function to integrate metabolic and immune signaling.

The ability of LXRs to regulate both lipid homeostatic and innate immune responses is particularly relevant in the context of atherogenesis. The present results indicate that the ability of oxysterols to antagonize LPS responses is mediated by LXRs. In contrast, the stimulatory effects of oxidized lipids on inflammatory gene expression in resting macrophages are likely to be mediated by different pathways, as LXR agonists did not alter expression of inflammatory targets in the absence of LPS. Thus, ligand activation of LXRs in lesion macrophages is envisioned to limit production of inflammatory molecules that exacerbate lesion development (Glass, C. K. &: Witztum, J. L. 2001 Cell 104:503-516; Lusis, A. I. 2000 Nature 407:233-241). In support of this idea, we found that treatment with LXR ligand reduced the expression of MMP-9 in the aortas of 8-month-old Apoe^{-/-} mice.

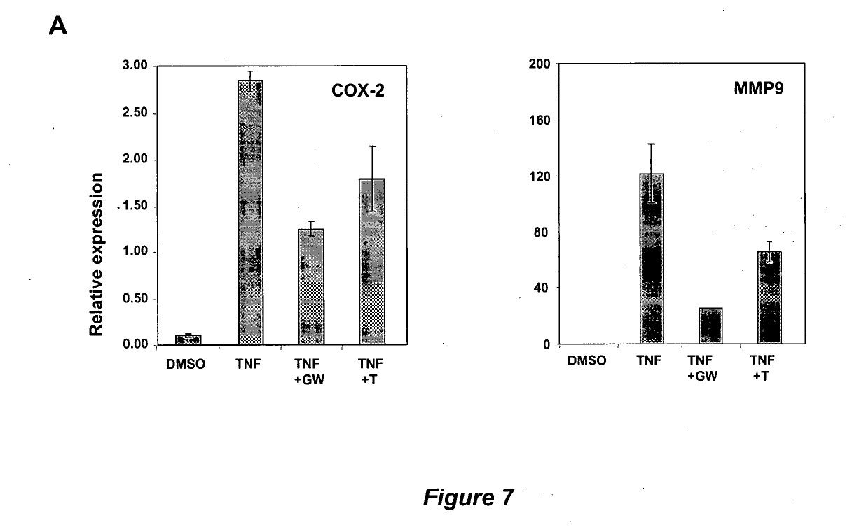

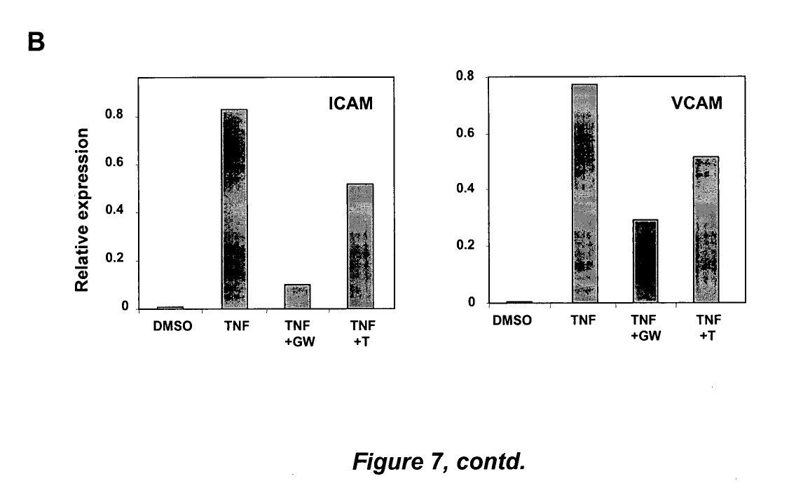

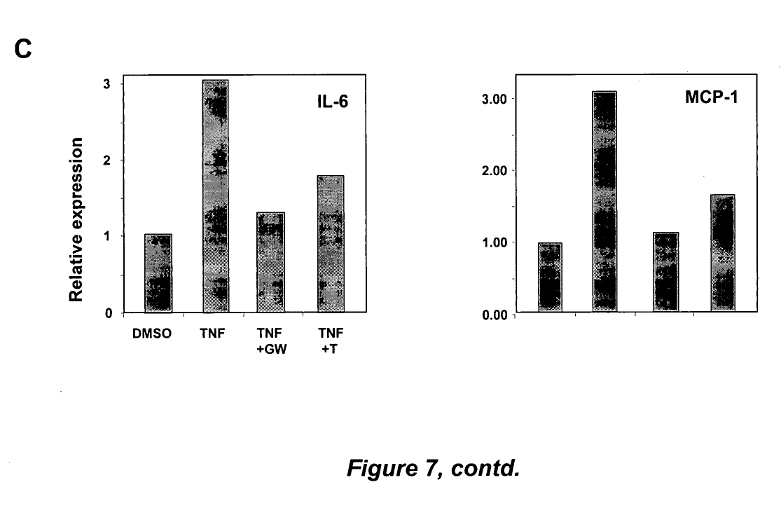

Activation of LXR by synthetic ligands inhibits inflammatory gene expression in multiple human cell types (FIG. 7A-C). TNFα plays a role in the induction of inflammatory mediators in during inflammation. Cells were treated for 6 hours with TNF as indicated and gene expression was measured by real time qPCR. Two different synthetic LXR ligands, GW3965 (GW) and T1317 (T) inhibited TNF-induced gene expression in human smooth muscle cells (A), human aortic endothelial cells (B), and human peripheral blood mononuclear cells (lymphocytes and monocytes, C). These data identify LXR as an anti-inflammatory target in many different cell types and indicate that LXR ligands are useful for the treatment of many different inflammatory diseases involving many different tissues.

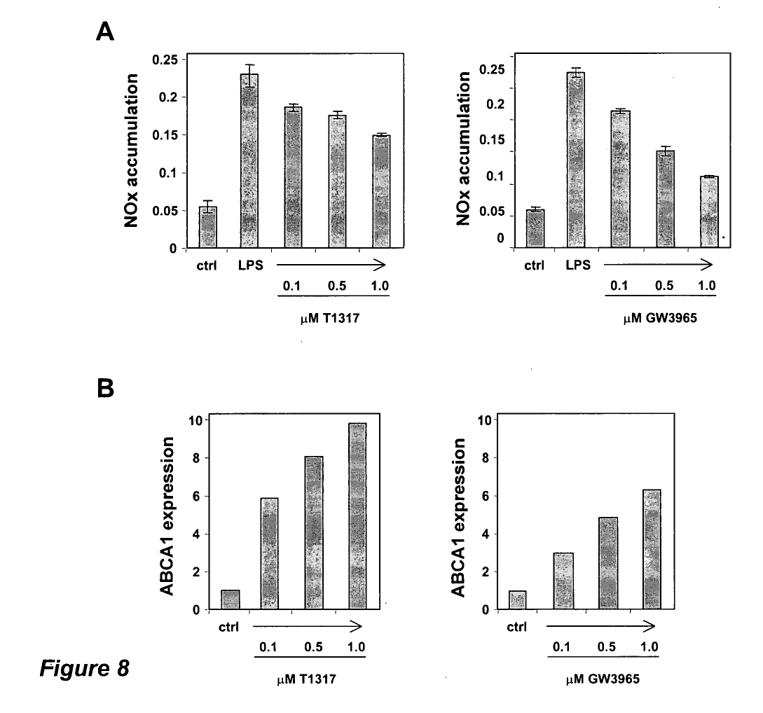

The anti-inflammatory activity of LXR ligands can be separated from the induction of lipid metabolism genes such as ABCA1. Murine peritoneal macrophages were treated with LPS to induce iNOS gene expression (FIG. 8A) and were assayed for ABCA1 mRNA by real time qPCR (FIG. 8B). NOx production was measured by colorimetric assay. In both experiments, cells were treated with increasing doses of two structurally different LXR ligands, T1317 and GW3965 as indicated. The data show that T1317 is a more effective activator of ABCA1 gene expression, whereas GW3965 is a more effective inhibitor of inflammatory genes. These data indicate that the two activities of LXR can be separated through the design of selective compounds. The identification of LXR agonists with only anti-inflammatory activity should greatly increase the usefulness of such compounds as therapeutic agents, because they lack the unwanted side effects on lipid metabolism such as elevation of triglyceride levels.

The approach to identification of selective anti-inflammatory ligands is based on the simultaneous assay of compound libraries for their effects in two different high throughput assays. The first assay is activation of an ABCA1, FAS or LXRE-driven reporter gene in an appropriate cell line. The second is an inflammation assay, such as for example, any of the following assays: NO production assay (as shown in FIGS. 1a and 8A), ELISA assay for expression of inflammatory proteins (e.g. IL-6), repression of NF-kB reporter gene in transfection assay, ELISA for prostaglandin production from cells. Macrophages are among the preferred cells to use for such a screening assay because of their role in inflammation. However, other cell types could also be used in a high throughput assay as long as they have an inflammatory response that can be readily measured. Compounds are selected based on their ability to act differentially in the two assays. Compounds with potent anti-inflammatory activity but reduced activation of the ABCA1 or LXRE reporter are the preferred target anti-inflammatory agents sought to be identified by the screening assay of the present invention.

### Methods

Reagents and plasmids. GW3965 (Collins J. L. et al 2002 J Med Chem 45:1963-1966), GW1516 (Oliver W R Jr. et al. 2001 PNAS USA 98:5306-5311), GW7647 (Oliver W R Jr. et al. 2001 PNAS USA 98:5306-5311), and T0901317 (Schultz J. R. et al. 2000 Genes Dev 14:2831-2838) were from GlaxoSmithKline. Oxysterols, LDL and ox-LDL were from Sigma and Intracell, respectively. LPS from Salmonella typhimurium was from Sigma. Expression plasmids for LXRα, LXRβ and IKK-DN (Castrillo, A. et al. 2001 J Biol Chem 276:15854-15860; Laffitte, B. A. et al. 2001 Mol Cell Biol 21:7558-7568), and reporter plasmids for NOS-2, (NFκB)₃-LUC, (AP-1)₄-LUC and COX-2 have been described (Callejas, N. A. et al. 2000 Gastroenterology 119:493-501).

Cell culture and transfections. Thioglycolate-elicited peritoneal macrophages were cultured in RPMI containing 10% fetal bovine serum (FBS). RAW 264.7 cells were cultured in DMEM containing 10% FBS. For ligand treatments, cells were in cultured medium supplemented with 5% lipoprotein-deficient serum (LPDS) and ligands for 18 h before stimulation. Bacterial infection in vitro was performed as described (Chen, L. M. et al. 1996 Mol Microbiol 21:1101-1115). Transient transfections of RAW 264.7 cells were performed in triplicate in 48-well plates using Superfect reagent (Life Technologies). After transfection, cells were incubated in medium containing 10% LPDS and the indicated ligands for 18 h before stimulation with LPS for an additional 18 h. Luciferase activity was normalized to β-galactosidase activity. NO production was determined spectrophotometrically (Castrillo, A. et al. 2000 Mol Cell Biol 20:1692-1698).

RNA and protein analysis. Total RNA was extracted using Trizol reagent (Life Technologies, Inc.). Northern blot analysis was carried out as described using radiolabeled cDNA probes (Tontonoz, P. et al. 1998 Cell 93:241-252). Real-time quantitative PCR (TaqMan) analysis was performed using an Applied Biosystems 7700 sequence detector. Two-step real-time PCR was performed as described (Joseph, S. B. et al. 2002 J Biol Chem 277:11019-11025). The primers and probes used are as follows:

| | |
|---|---|
| 36b4 cDNA-189 Forward primer: AGATGCAGCAGATCCGCAT; | (SEQ ID NO: 1) |
| 36b4 cDNA-247 Reverse primer: GTTCTTGCCCATCAGCACC; | (SEQ ID NO: 2) |
| 36b4 Taqman probe: FAMCGCTCCGAGGGAAGGCCG; | (SEQ ID NO: 3) |
| mABCA1 cDNA-6484 Forward primer: GGTTTGGAGATGGTTATACAATAGTTGT; | (SEQ ID NO: 4) |
| mABCA1 cDNA-6577 Reverse primer: CCCGGAAACGCAAGTCC; | (SEQ ID NO: 5) |
| mABCA1 Taqman probe: (6513): FAM-CGAATAGCAGGCTCCAACCCTGACC; | (SEQ ID NO: 6) |
| mTNFα cDNA-243 Forward primer: ATCATCTTCTCAAAACTCGAGTGA; | (SEQ ID NO: 7) |
| mTNFα cDNA-368 Reverse primer: TTGAGATCCATGCCATTGG; | (SEQ ID NO: 8) |
| mTNFα Taqman probe: (276+) FAM-AGCCCACGTCGTAGCAAACCACC; | (SEQ ID NO: 9) |
| MMP-9 Forward primer: 5′-TCACCTTCACCCGCGTGTA-3′; | (SEQ ID NO: 10) |
| MMP-9 Reverse primer: 5′-GTCCTCCGCGACACCAA-3′; | (SEQ ID NO: 11) |
| MMP-9 probe: 5′-/56-FAM-ACCCGAAGCGGACATTGTCATCCAG/3BHQ-1/-3′; | (SEQ ID NO: 12) |
| iNOS Forward primer:GCAGCTGGGCTGTACAAA; | SEQ ID NO: 13) |
| iNOS Reverse primer: AGCGTTTCGGGATCTGAAT; | (SEQ ID NO: 14) |
| iNOS Probe: FAM-TCAAAGGTCTCACAGGCT; | (SEQ ID NO: 15) |
| IL-6 Forward primer: CTGCAAGAGACTTCCATCCAGTT; | (SEQ ID NO: 16) |
| IL-6 Reverse primer: GAAGTAGGGAAGGCCGTGG; | (SEQ ID NO: 17) |
| IL-6 Probe: FAM-TGTCACCAGCATCAGTCCCAAGAAGG; | (SEQ ID NO: 18) |
| IL-1β Forward primer: AGAAGCTGTGGCAGCTACCTG; | (SEQ ID NO: 19) |
| IL-1β Reverse primer REV:GGAAAAGAAGGTGCTCATGTCC; | (SEQ ID NO: 20) |
| IL-1β probe: FAM-TCTTTCCCGTGGACCTTCCAGGATG; | (SEQ ID NO: 21) |
| IL-1-RN Forward primer: TGTTTAGCTCACCCATGGCTT; | (SEQ ID NO: 22) |
| IL-1-RN Reverse primer: TCTTGCAGGGTCTTTTCCCA; | (SEQ ID NO: 23) |
| IL-1-RN Probe: FAM-AGAGGCAGCCTGCCGCCCTT; | (SEQ ID NO: 24) |
| CD-68 Forward primer: CAAGGTCCAGGGAGGTTGTG; | (SEQ ID NO: 25) |
| CD-68 Reverse primer: CGGTACCCATCCCCACCTGTCTCTCTC; | (SEQ ID NO: 26) |
| CD-68 Probe: CCAAAGGTAAGCTGTCCATAAGGA; | (SEQ ID NO: 27) |
| MCP-1 Forward primer: AGAAGCTGTGATCTTCAAGACCATT; | (SEQ ID NO: 28) |
| MCP-1 Reverse primer: TGCTTGTCCAGGTGGTCCAT; | (SEQ ID NO: 29) |
| MCP-1 Probe: FAM-CCAAGGAGATCTGTGCTGACCCCAA. | (SEQ ID NO: 30) |

Reactions were performed in duplicate and normalized to 36B4 expression. Western blot analysis was performed as described (Castrillo, A. et al. 2001 J Biol Chem 276:15854-15860). Antibodies against NOS-2 (SC-651) and COX-2 (160106) were from Santa Cruz Biotechnology and Cayman Chemicals, respectively.

DNA microarray analysis. Thioglycolate-elicited peritoneal macrophages were cultured in DMEM/10% LPDS and treated with vehicle or 2 μM GW3965 for 18 h before stimulation with 100 ng/ml LPS for 6 h. Total RNA was reverse transcribed using a T7-(dT)₂₄ primer (Genset Corp.). Biotin-labeled cRNA was generated using the Bioarray High Yield Transcript Labeling Kit (ENZO). Samples were hybridized to single Affymetrix Murine U74Av2 microarrays and visualized by the PAN Facility at Stanford University. Data was analyzed using GeneSpring and GeneChip Analysis Suite software (Affymetrix). Only those results determined by the software to represent significant expression differences in the presence or absence of ligand are presented. Positive results were defined as ligand-dependent changes in expression in wild-type cells.

Animals. Mice on a mixed background (C57B1/6 and 129v) and Apoe^{-/-} mice on a C57B1/6 background were maintained on standard chow. Where indicated, diets were supplemented with GW3965 at a level sufficient to provide a 20 mg/kg body weight dose per day. Irritant contact dermatitis was induced as described (Sheu, M. Y. et al. 2002 J Invest Dermatol 118:94-101). Briefly, 10 μl of 0.03% 12-O-tetradecanoylphorbol-13-acetate (TPA) in acetone was applied to the left ears of male mice. The right ears were treated with vehicle alone. We applied 20 μl of 10 mM T1317, 10 mM GW3965, or vehicle to both left and right ears at 45 min and 4 h post-TPA treatment. After 18 h of TPA treatment, the thickness and weight of 6-mm punch biopsies were determined. Statistical analysis was performed using Student's t-test. Experiments were conducted in accordance of the Animal Research Committee of the University of California, Los Angeles.

While the present invention has been described in some detail for purposes of clarity and understanding, one skilled in the art will appreciate that various changes in form and detail can be made without departing from the true scope of the invention. All figures, tables, and publications, referred to above, are hereby incorporated by reference.

What is claimed is:

1. A method of reducing of inflammation in a mammal, comprising administering to the mammal a therapeutically effective amount of an LXR agonist.

2. A method of reducing inflammation in a mammal, comprising administering to the mammal a therapeutically effective amount of an LXR agonist, wherein said therapeutically effective amount of said LXR agonist exhibits a relatively greater effect on expression of an inflammatory gene than on expression of a lipid metabolism gene, such that inflammation is reduced without a significant effect on lipid metabolism.

3. A method for treating an inflammatory disease in a subject, comprising administering to the subject a therapeutically effective amount of an LXR agonist.

4. A method for treating, controlling, preventing or reducing the risk of contracting an inflammatory disease or condition in a mammal comprising
selecting a mammal in need thereof, and
administering to said mammal a therapeutically effective amount of an LXR agonist.

5. The method of claim 4, wherein the inflammatory disease or condition is selected from the group consisting of rheumatoid arthritis, juvenile rheumatoid arthritis, systemic lupus erythematosus, osteoarthritis, degenerative joint disease, one or more connective tissue diseases, ankylosing spondylitis, bursitis, Sjogren's syndrome, psoriasis, psoriatic arthritis, neuralgia, synoviitis, glomerulonephritis, vasculitis, sarcoidosis, atherosclerosis, asthma, inflammatory bowel disease, inflammations that occur as sequellae to influenza, the common cold and other viral infections, gout, contact dermatitis, low back and neck pain, dysmenorrhea, headache, toothache, sprains, strains, myositis, burns, injuries, and pain and inflammation that follow surgical and dental procedures in a patient.

6. A method for treating, controlling, preventing or reducing the risk of contracting an inflammatory disease or condition in a mammal, comprising
selecting a mammal in need thereof, and
administering to the mammal a therapeutically effective amount of an LXR agonist and one or more additional therapeutic compounds, wherein said additional therapeutic compounds are selected from the group consisting of a pain reliever, an NSAID, a corticosteroid, a caffeine, an H2-antagonist, aluminum or magnesium hydroxide, simethicone, a decongestant, an antitussive, a diuretic, and a sedating or non-sedating antihistamine.

7. The method of claim 6, wherein said pain reliever is selected from the group consisting of acetominophen and phenacetin; said NSAID is a non-selective or selective COX-2 inhibitor, said corticosteroid is selected from the group consisting of hydrocortisone, prednisolone, 6-alpha-methylprednisolone, triamcinolone, dexamethasone and betaroethasone, said decongestant selected from phenylephrine, phenylpropanolamine, pseudoephedrine, oxymetazoline, epinephrine, naphazoline, xylometazoline, propylhexedrine, and levo-desoxyephedrine; said antitussive is selected from the group consisting of codeine, hydrocodone, caramiphen, carbetapentane, and dextramethorphan.

8. A method for treating, controlling, preventing or reducing the risk of contracting an inflammatory disease or condition in a mammal, comprising
selecting a mammalian patient in need thereof; and
administering to the mammal a therapeutically effective amount of an LXR agonist and an NSAID.

9. The method of claim 8, wherein said NSAID is a non-selective COX-2 inhibitor.

10. The method of claim 8, wherein said NSAID is a selective COX-2 inhibitor.

11. The method of claim 10, wherein said selective COX-2 inhibitor is selected from the group consisting of rofecoxib, etoricoxib, celecoxib, parecoxib and valdecoxib.

12. A method for treating, controlling, preventing or reducing the risk of contracting an inflammatory disease or condition in a mammal, comprising
selecting a mammal in need thereof; and
administering to the mammal a therapeutically effective amount of an LXR agonist and a corticosteroid.

13. The method of claim 12, wherein said corticosteroid is selected from the group consisting of hydrocortisone, prednisolone, 6-alpha-methylprednisolone, triamcinolone, dexamethasone and betaroethasone.

14. A method for treating an inflammatory disease in a subject, comprising administering to the subject a therapeutically effective amount of an LXR agonist, wherein said inflammatory disease is macrophage-dependentt.

15. The method of claim 1, wherein said inflammatory disease is atherosclerosis, rheumatoid arthritis, or glomerulonephritis.

16. A method for inhibiting inflammatory gene expression in a mammal, comprising administering to the mammal an amount of an LXR agonist sufficient to antagonize NF-κB signaling such that said inflammatory gene expression is inhibited.

17. The method of any one of claims 1, 2, 3, 4, 5, 6, 7, 9, 11 13, 14, or 16, wherein said LXR agonist is GW3965 or T0901317.

18. A screening method for identifying an anti-inflammatory compound, comprising:
(a) treating a cell with a test compound;
(b) measuring expression in said cell of a gene involved in lipid metabolism in response to said test compound;
(c) measuring expression in said cell of a gene involved in inflammation in response to said test compound;
(d) repeating steps (a)-(c) with another test compound; and
(e) identifying the compound which preferentially alters the expression of the gene involved in inflammation.

19. A method of reducing of inflammation in a mammal, comprising
administering to the mammal a therapeutically effective amount of the compound identified by the screening method of claim 18.

20. A pharmaceutical composition, comprising a first agent comprising the anti-inflammatory compound identified by the screening method of claim 18 and a second agent selected from the group consisting of a NSAID and a glucocorticoid, wherein therapeutically effective amounts of the first and second agents are formulated in a pharmaceutically acceptable carrier.
